(19) United States
(12) Patent Application Publication
(10) Pub. No.: US 2014/0039051 A1
(43) Pub. Date: Feb. 6, 2014

(54) Cleaning and disinfection agent for medical instruments

(71) Applicant: Chemische Fabrik Dr. Weigert GmbH & Co. KG, Hamburg (DE)

(72) Inventors: Iris Strodtholz, Hamburg (DE); Verona Schmidt, Hamburg (DE); Markus Kamer, Reinbek (DE)

(73) Assignee: Chemische Fabrik Dr. Weigert GmbH & Co. KG, Hamburg (DE)

(21) Appl. No.: 13/956,751

(22) Filed: Aug. 1, 2013

(30) Foreign Application Priority Data
Aug. 1, 2012 (EP) 12178831.9

(51) Int. Cl. A61L 2/23

(52) U.S. Cl. CPC A61L 2/23
USPC 514/557; 510/161; 134/42

(57) ABSTRACT

The invention relates to a cleaner and/or disinfectant for medical and/or surgical elements and apparatuses which is formulated as a powder. It comprises at least one peroxide, at least one acylating agent for releasing peracetic acid from the peroxide in aqueous solution, and also at least one nonionic surfactant. According to the invention, it is provided that it comprises agents for adjusting a pH of a 2% strength aqueous solution to pH 7.5 to 9 and is formulated as granules, where peroxide and/or acylating agent are coated with nonionic surfactant.

The invention relates to a cleaner and/or disinfectant for medical and/or surgical elements and apparatuses which is formulated as a powder. It comprises at least one peroxide, at least one acylating agent for releasing peracetic acid from the peroxide in aqueous solution, and at least one nonionic surfactant.

Disinfectants for medical and/or surgical instruments and apparatuses are widespread in practice. The disinfection effect is often based on aldehydes, quaternary ammonium compounds, phenols, alcohols or other active ingredients.

Cleaners and disinfectants based on peroxides, in particular peracetic acid, are likewise known. Peroxides are readily antimicrobially effective, but generally do not have very good storage stability.

EP 1 489 908 A1 already discloses a composition according to the preamble of claim 1. Compared with this prior art, the object of the invention is to provide a cleaner and/or disinfectant of the type specified at the start which exhibits good storability and rapidly develops its full spectrum of activity following dissolution in water.

According to the invention, this object is achieved by virtue of the fact that it comprises agents for adjusting a pH of a 2% strength aqueous solution (all data within the context of the invention are % by weight) to pH 7.5 to 9 and is formulated as granules, where peroxide and/or acylating agent are coated with nonionic surfactant.

Firstly, some of the terms used in the scope of the invention are explained.

The term powder refers to sprinklable and/or pourable solids, including granules.

Cleaners and/or disinfectants for medical and/or surgical elements and apparatuses are those compositions which at least reduce the soiling and in particular germ contamination in the course of processing such instruments. The invention is particularly suitable in the course of the cleaning and/or disinfection of endoscopes.

Acylating agents are those compounds which are able to release peracetic acid from the peroxide in aqueous solution. These may be, for example, acyloxycarboxylic acids, but in particular N-acyl compounds.

The agents for establishing the pH intended according to the invention of 7.5 to 9 can include in particular suitable acids and/or buffer systems. The customary application concentration at which the pH should be adjusted is a solution of 2% by weight of the powder in water. According to the invention, it may be provided and preferred that this pH is also established for a 1% strength solution and the concentrations in between.

Within the context of the invention, the term granules refers to a pourable solid in which peroxide, acylating agent, optionally also other constituents such as for example the agents for establishing the pH are coated with the nonionic surfactant. This means that in any case a considerable part of the nonionic surfactant is enriched in the region of the surfaces of the granule particles. The coating can be accomplished by suitable processes known to the person skilled in the art, such as, for example, spraying-on. Nonionic surfactants used according to the invention are sufficiently liquid or flowable at room temperature or after slight heating to, for example, 30 to 40° C. in order to enable them to be sprayed.

According to the invention, the grain size of the granules can be preferably in the range 0.1 to 2 mm, further preferably 0.2 to 1.6 mm, further preferably 0.4 to 1.2 mm. Preferably at least 80% by weight, further preferably at least 90% by weight, of the granules then fall within the respective size range.

Surgical instruments such as, in particular, endoscopes are often firstly prewashed manually after use before they are subjected to a complete processing cycle. Usually, precleaning is often directly following use, the aim being to reduce germ contamination to the extent that the risk to personnel handling decontaminated instruments is minimized. Alternatively or additionally, a manual disinfection in an immersion bath is often also practiced.

Both cases rely on the fact that after preparing a corresponding aqueous solution from the solid, an antimicrobial effect rapidly occurs, i.e. an effective concentration of free peracetic acid is rapidly established in the aqueous solution. The invention has recognized that by coating the granules with the nonionic surfactant, a rapid decomposition of the granules and the physical dissolution process are promoted. The adjustment of the pH to the claimed range 7.5 to 9 in the ready-to-use diluted solution additionally contributes to the fact that the acylating agent rapidly releases sufficient amounts of peracetic acid from the peroxide and consequently, at the latest after 15 minutes, a peracetic acid concentration adequate for the application purposes and thereby effective is present in the aqueous solution. According to the invention, the pH is particularly preferably adjusted to the range 7.5 to 8.5, further preferably 7.6 to 7.9.

The coating of the granules with the nonionic surfactant also contributes to the fact that the disintegration reaction that takes place during the storage of a mixture of acylating agents and peroxides is minimized, and therefore the storability of the composition according to the invention is increased.

The invention therefore provides a composition which can be stored for at least one year, preferably at least two years, upon customary storage at room temperature, preferably also at 40° C., and which rapidly (within 15 min) makes available an effective concentration of free peracetic acid following dissolution in water. Typical application concentrations of a composition according to the invention in aqueous solution are 1 or 2% by weight. In a 1% strength solution, a concentration of free peracetic acid of at least 900 ppm, typically ca. 1500 ppm, is typically established after 15 min. For a 2% strength solution, it is at least 2600 ppm, typically 3000 ppm. The peroxides used according to the invention are preferably inorganic peroxides such as, in particular, perborates or percarbonates. Preference is given to the sodium salts. Particular preference is given to sodium percarbonate. According to the invention, it is possible and preferred that the peroxide used is likewise provided with a coating prior to the mixing in order to minimize undesired decomposition reactions upon storage together with the acylating agent. For example, the peroxide can be provided with an oleate/sodium sulfate coating.

According to the invention, the acylating agent can be selected from the group consisting of tetraacetylethylenediamine (TAED), tetraacetylglycoluril and diacetylhexahydrotriazinedione. Tetraacetylethylenediamine (TAED) is particularly preferred.

According to the invention, the nonionic surfactants can be selected from the group consisting of fatty alcohol ethoxylates, fatty alcohol propoxylates, EO-PO block copolymers, alkyl glucosides, alkyl polyglucosides, octylphenol ethoxylates and nonylphenol ethoxylates. Ethoxylated fatty alcohols known to the person skilled in the art are particularly preferred.

The agents envisaged for adjusting the pH are preferably acids and/or a buffer system. Particular preference is given to citric acid (preferably the anhydrous anhydride), citrate buffer, phosphate buffer or carbonate buffer. According to the invention, citric acid can be combined for example with a phosphate, preferably sodium tripolyphosphate.

The composition according to the invention is formulated as a solid. Preferably, it is essentially anhydrous in order to minimize decomposition reactions of the peroxide and to increase the storage stability. The sum of the mass fractions of peroxide, acylating agent and agent for adjusting the pH constitutes preferably at least 60% by weight, further preferably at least 70% by weight, further preferably at least 80% by weight, further preferably at least 90% by weight, of the total mass of the composition according to the invention. This sum can constitute for example 95% by weight of the total mass of the composition.

Within the context of the invention, preference is given to the following weight fractions of the ingredients, based on the total mass of the composition formulated as a solid:
peroxide 40-70, preferably 50-60% by weight;
acylating agent 15-40, preferably 20-30% by weight,
agent for adjusting the pH 5-25% by weight, preferably 10-20% by weight,
nonionic surfactant 1-5% by weight, preferably 2-4% by weight.

The invention further provides a process for producing a composition according to the invention. In this process, firstly peroxide, acylating agent and agent for adjusting the pH (if appropriate also further optional ingredients) are mixed together. Granulation is then carried out while spraying on the liquid nonionic surfactant. The coating can be accomplished for example by means of a rotary spray mixer. Typical spray parameters that can be used according to the invention are a spraying pressure of 4 bar and a spraying output of 175 g/s.

The aforementioned optional further additives may be, for example, enzymes, in particular proteolytic enzymes, further surfactants or the like. These ingredients can contribute to improving the cleaning performance of the composition according to the invention.

The invention further provides a method for the cleaning and/or disinfection of medical and/or surgical instruments and apparatuses (in particular endoscopes), which involves the following steps:
a) producing a 0.5 to 3% strength, preferably 1 to 2% strength, aqueous solution of a cleaner and/or disinfectant as claimed in any one of claims 1 to 9,
b) cleaning and/or disinfecting the medical and/or surgical instruments and apparatuses.

The application concentration can be dependent on the envisaged intended use. If it is desired to merely carry out a precleaning to reduce the germ contamination, a disinfection in a 1% strength solution over a period of 5 min may be sufficient, for example. Should complete cleaning and disinfection (preferably manually) take place, according to the invention, cleaning and disinfection is preferably carried out with a 2% strength immersion bath over a period of 15 min.

According to the invention, this preferably takes place in an immersion bath.

In the case of manual application, the temperature of the solution during application is preferably about 20° C. (room temperature) to 40° C.

The cleaning and/or disinfection takes place preferably over a period of 5 to 30 min, further preferably 10 to 20 min.

Working examples of the invention are described below.

### EXAMPLE 1

To prepare a cleaner and disinfectant according to the invention, the following starting materials are used in the stated weight fractions:

| | % by wt. |
|---|---|
| Na percarbonate (Harke Chemicals) | 55.0 |
| TAED (Peractive ® P, Clariant) | 25.0 |
| Na tripolyphosphate (provided in sacks, | 2.0 |
| coarse 850-1100, BK Giulini) | |
| Citric acid anhydr. | 15.0 |
| PPG-4 Laureth-5 (fatty alcohol C10/12, | 3.0 |
| 5 EO/4 PO, Cognis) | |

The powder constituents (all of the constituents apart from the nonionic surfactant) are introduced with stirring into a rotary mixer and uniformly mixed. The nonionic surfactant is heated to 40° C. and sprayed onto the powder mixture, as mixing is continued, within 4 min at a spraying pressure of 4 bar. Mixing is then carried out for about a further 12 min.

### EXAMPLE 2

The procedure is as in example 1, the starting materials used being as follows:

| | % by wt. |
|---|---|
| Na percarbonate (Harke Chemicals) | 49.0 |
| TAED (Peractive ® AN, peroxide with an | 29.0 |
| oleate/sodium sulfate coating, Clariant) | |
| Na tripolyphosphate (provided in sacks, coarse | 4.0 |
| 850-1100, BK Giulini) | |
| Citric acid anhydr. | 12.0 |
| Dehypon ® GRA (modified fatty alcohol polyglycol | 4.0 |
| ether, BASF) | |
| K carbonate (anhydrous, ca. 95% strength) | 2.0 |

In this example, a different nonionic surfactant is used and the buffer system is modified by using potassium carbonate.

### EXAMPLE 3

The procedure is as in example 1, the starting materials used being as follows:

| | % by wt. |
|---|---|
| Na percarbonate (Harke Chemicals) | 50.0 |
| TAED (Peractive ® AN, peroxide with an | 29.0 |
| oleate/sodium sulfate coating, Clariant) | |
| Na tripolyphosphate (provided in sacks, coarse | 3.0 |
| 850-1100, BK Giulini) | |
| Citric acid anhydr. | 15.0 |
| Fatty alcohol C10, 11 EO | 3.0 |
| (Lutensol ® ON110, BASF) | |

In this example, a further nonionic surfactant is used.

### EXAMPLE 4

The procedure is as in example 1, the starting materials used being as follows:

| | % by wt. |
|---|---|
| Na percarbonate (Harke Chemicals) | 50.0 |
| TAED (Peractive ® AN, peroxide with an | 29.0 |
| oleate/sodium sulfate coating, Clariant) | |
| Esperase (Novozymes) | 1.0 |
| Citric acid anhydr. | 15.0 |
| Fatty alcohol C10/12, 10 EO | 2.0 |
| (Dehypon ® LS 104 1, BASF) | |
| K carbonate (anhydrous, ca. 95% strength) | 3.0 |

In this example, a different nonionic surfactant is again used, and also a proteolytic enzyme is additionally present.

### EXAMPLE 5

In this example, a precleaning or predisinfection (partial disinfection to the extent that the risk of contamination for people subsequently handling the endoscopes is minimized) is carried out.

In a cleaning tub, 1% by weight of example 1 is dissolved in lukewarm water. After 15 min, a concentration of ca. 1500 ppm of free peracetic acid is established. According to the invention, this concentration should as far as possible be 900 ppm or above.

Directly after the investigation, the endoscope (which is still attached to the light source and the suction pump) is wiped with a lint-free cloth still in the investigation room. Drying-on of the organic residues should be avoided. The endoscope is then dipped into the solution and all of the cannulae are flushed through or sucked through several times. The endoscope is separated from the light source and the suction apparatus, the cleaning tub is sealed and transported to the processing room.

### EXAMPLE 6

In this example, a manual cleaning and disinfection of endoscopes is carried out.

In a cleaning tub, 2% by weight of example 1 are dissolved in lukewarm water. After 15 min, a concentration of ca. 3000 ppm of free peracetic acid is established. The endoscope or the accessory instruments are placed into the solution such that all of the surfaces are completely wetted and no air bubbles are present. All of the cleaning steps are carried out below the surface of the liquid. The endoscope cannulae are carefully cleaned with brushes, using a suitably sized disinfected brush for each cannula and observing the instructions of the endoscope manufacturer. To rinse off the application solution, in each case fresh, microbiologically unobjectionable water should be used, it being necessary to carefully rinse all of the cannulae and the outer casing of the endoscope.

### EXAMPLE 7

It is important for effective disinfection that, after preparing the solution from the composition according to the invention, a sufficient concentration of free peracetic acid is rapidly established and is retained in the solution over a prolonged application period.

A solution of 2% by weight of the composition of example 1 in town water is prepared (stirring for 15 min at 25° C.) The concentration of the free peracetic acid in the solution is determined, the determination is repeated at the time intervals shown in table 2. It can be seen that an effective concentration is established directly after preparing the solution and is retained over a customary application period of 8 h.

TABLE 2

| Time [h] | Peracetic acid [ppm] |
|---|---|
| 0 | 3345 |
| 1 | 2991 |
| 2 | 2631 |
| 3 | 2564 |
| 4 | 2354 |
| 5 | 2106 |
| 6 | 2054 |
| 7 | 1880 |
| 8 | 1591 |

### EXAMPLE 8

To test the storability of the composition according to the invention, in each case 2 kg of the powder in example 1 are placed into a 3 l bucket (polypropylene) and the bucket is closed with a lid. The samples thus prepared were exposed as follows to different storage conditions over a period of a total of twelve weeks:
6° C., 55% relative humidity
21° C., 55% relative humidity
30° C., 70% relative humidity
40° C., 75% relative humidity
alternating climate 6° C., 21° C., 40° C., 21° C., 6° C. each for 5 h with a 1 h transition time in each case between the temperatures

Each sample was assessed after storage for 4, 8 and 12 weeks as to appearance of powder or granules and 2% strength aqueous solution prepared therefrom, pH of the solution and concentration of free peracetic acid in the solution.

For all of the samples and all of the storage conditions, the results were as follows:

The granules retained their original white-yellowish color and pourability. The aqueous solution was clear and colorless. The content of free peracetic acid in the aqueous solution after stirring for 15 min was between 2600 and 3000 ppm. The pH of the solution was between 7.6 and 7.8.

These storage experiments lead to the conclusion that a composition according to the invention is storable at room temperature for at least 2 years.

### EXAMPLE 9

In this example, the cleaning effect of a composition according to the invention is investigated. It is a primary aim of the invention to effect disinfection through the release of free peracetic acid in the aqueous solution. The thorough and good disinfection effect of peracetic acid has been known for a long time in the prior art.

Additionally, the composition according to the invention should advantageously likewise contribute to cleaning. The immersion experiments carried out below are intended to quantify this cleaning performance. The experiment is designed (simple immersion into the solution without direct mechanical action on the soiled surfaces) such that complete removal of the test soiling is not possible and consequently can also not be expected.

The soiling carriers used were rough metal plates measuring 5×10 cm. 200 μl of defibrinated sheep blood (Acila Ch. B. 24632) were applied to the cleaned and weighed metal plates and dried overnight at room temperature. The test plates were then weighed.

In a 600 ml beaker, 500 g of 2% strength aqueous solution of example 1 were prepared. The solution was stirred while carrying out the experiment at room temperature using a magnetic stirrer (setting 8).

The test plates were immersed into the solution for 15 min and then briefly rinsed by dipping them into demineralized water. After drying overnight, the test plates were weighed again.

A residual amount of 48.6% by weight of the test soiling remained on the test plates after this experiment. In the case of standard commercial disinfectants for endoscopes that were tested for comparison purposes, the residual amount was significantly higher (up to 96.9% by weight). This shows that the composition according to the invention not only brings about good disinfection, but also contributes substantially to the cleaning.

1. A cleaner and/or disinfectant formulated as powder for cleaning and/or disinfecting medical and/or surgical instruments and apparatuses, comprising:
a) at least one peroxide,
b) at least one acylating agent for releasing peracetic acid from the peroxide in aqueous solution,
c) at least one nonionic surfactant,
wherein said powder has the following features:
d) it comprises agents for adjusting a pH of a 2% strength aqueous solution to pH 7.5 to 9,
e) it is formulated as granules, which peroxide and/or acylating agent are coated with nonionic surfactant.

2. The cleaner and/or disinfectant as claimed in claim 1, wherein the peroxide comprises a perborate and/or percarbonate.

3. The cleaner and/or disinfectant as claimed in claim 1, wherein the acylating agent is selected from the group consisting of tetraacetylethylenediamine (TAED), tetraacetylglycoluril and diacetylhexahydrotriazinedione.

4. The cleaner and/or disinfectant as claimed in claim 1, wherein the nonionic surfactants are selected from the group consisting of fatty alcohol ethoxylates, fatty alcohol propoxylates, EO-PO block copolymers, alkyl glucosides, alkyl polyglucosides, octylphenol ethoxylates and nonylphenol ethoxylates.

5. The cleaner and/or disinfectant as claimed in claim 1, wherein said cleaner and/or disinfectant comprises an acid and/or a buffer system as said agent for adjusting the pH.

6. The cleaner and/or disinfectant as claimed in claim 5, wherein said cleaner and/or disinfectant comprises citric acid and/or a phosphate buffer or carbonate buffer as agent for adjusting the pH.

7. The cleaner and/or disinfectant as claimed in claim 6, wherein said cleaner and/or disinfectant comprises sodium tripolyphosphate as phosphate buffer.

8. The cleaner and/or disinfectant as claimed in claim 1, wherein the sum of peroxide, acylating agent and agent for adjusting the pH is at least 60% by weight of the total mass of the cleaner and/or disinfectant.

9. The cleaner and/or disinfectant as claimed in claim 1, which wherein said cleaner and/or disinfectant has the following weight fractions of the ingredients:
peroxide: 40 to 70% by weight;
acylating agent: 15 to 40% by weight,
agent for adjusting the pH: 5-25% by weight,
nonionic surfactant 1 to 5% by weight,

10.-15. (canceled)

16. The cleaner and/or disinfectant as claimed in claim 8, wherein the sum of peroxide, acylating agent, and agent for adjusting the pH is at least 70% by weight of the total mass of the cleaner and/or disinfectant.

17. The cleaner and/or disinfectant as claimed in claim 8, wherein the sum of peroxide, acylating agent, and agent for adjusting the pH is at least 80% by weight of the total mass of the cleaner and/or disinfectant.

18. The cleaner and/or disinfectant as claimed in claim 8, wherein the sum of peroxide, acylating agent, and agent for adjusting the pH is at least 90% by weight of the total mass of the cleaner and/or disinfectant.

19. The cleaner and/or disinfectant as claimed in claim 9, wherein the weight fraction of the ingredients of said peroxide is 50 to 60% by weight.

20. The cleaner and/or disinfectant as claimed in claim 9, wherein the weight fraction of the ingredients of said acylating agent is 20 to 30% by weight.

21. The cleaner and/or disinfectant as claimed in claim 9, wherein the weight fraction of the ingredients of said agent for adjusting the pH is 10 to 20% by weight.

22. The cleaner and/or disinfectant as claimed in claim 9, wherein the weight fraction of the ingredients of said nonionic surfactant is 2 to 4% by weight.

23. A process for producing a cleaner and/or disinfectant as claimed in claim 1, said process comprising the steps of:
a) mixing of peroxide, acylating agent and agent for adjusting the pH, and
b) carrying out a granulation with spraying-on of the nonionic surfactant.

24. A method for the cleaning and/or disinfection of medical and/or surgical instruments and apparatuses, which involves the following steps:
a) producing a 0.5 to 3% strength aqueous solution of a cleaner and/or disinfectant as claimed in claim 1, and
b) cleaning and/or disinfecting the medical and/or surgical instruments and apparatuses with said aqueous solution.

25. The method of claim 24, wherein said aqueous solution is 1 to 2% strength.

26. The method as claimed in claim 24, wherein the cleaning/disinfection takes place in an immersion bath.

27. The method as claimed in claim 24, wherein the cleaning/disinfection takes place at 20 to 40° C.

28. The method as claimed in claim 24, wherein the cleaning/disinfection takes place over a period of 5 to 30 min.

29. The method as claimed in claim 24, wherein the cleaning/disinfection takes place over a period of 10 to 20 min.

30. The method as claimed in 24, wherein said cleaning/disinfection takes place manually.
